(19) United States
(12) Patent Application Publication
(10) Pub. No.: US 2010/0074848 A1
(43) Pub. Date: Mar. 25, 2010

(54) X-ray contrast agent using gold nanoparticles and process for preparing the same

(76) Inventor: Kwon-ha Yoon, Jeollabuk-do (KR)

Correspondence Address: LRK Patent Law Firm, 1952 Gallows Rd, Suite 200, Vienna, VA, 22182, US

(21) Appl. No.: 12/300,336

(22) PCT Filed: Aug. 31, 2006
(86) PCT No.: PCT/KR2006/003452

§ 371 Date: Apr. 15, 2009

(30) Foreign Application Priority Data
May 9, 2006 (KR) 10-2006-0041593

(51) Int. Cl. A61K 49/04

(52) U.S. Cl. 424/9.42

(57) ABSTRACT

Provided are an X-ray contrast agent using gold nanoparticles and a process for preparing the X-ray contrast agent. The X-ray contrast agent is prepared by capping gold nanoparticles with polyethylene glycol (PEG). The X-ray contrast agent is nontoxic and remains in a blood vessel for a long time so that the X-ray contrast agent can be usefully used as a computed tomography (CT) contrast agent for imaging blood vessels. Therefore, the X-ray contrast agent is useful for inspecting blood vessel structures and new blood vessels in a tumor, and obtaining perfusion images. In addition, gold provides better visibility than iodine when used in an X-ray contrast agent. Therefore, better x-ray images can be obtained using the X-ray contrast agent containing gold nanoparticles.

### TECHNICAL FIELD

The present invention relates to an contrast agent and a process for preparing the contrast agent, and more particularly, to a nontoxic X-ray contrast agent capable of remaining in a blood vessel for a long time and formed using a gold nanoparticle colloid and a polyethylene compound to be used as a computed tomography (CT) contrast agent for obtaining blood vessel images, and a process for preparing the X-ray contrast agent.

### BACKGROUND ART

Most of conventional CT contrast agents are formed using iodine. However, since iodine is toxic, it is difficult to use such iodine contrast agents for a patient having a kidney disease. In addition, such iodine contrast agents cannot remain in a human body for a long time.

Moreover, the iodine contrast agents result in various toxic symptoms such as a pain, a hot flash, and an allergy. Particularly, the iodine contrast agents are very harmful to the biliary tree.

Most of commercialized CT contrast agents are formed using toxic iodine, and a CT contrast agent formed using nontoxic gold has not been developed.

### DISCLOSURE

### Technical Problem

An object of the present invention is to provide a nontoxic contrast agent formed using gold and capable of remaining in a human body for a long time, and a process of preparing the contrast agent.

Another object of the present invention is to provide a contrast agent suitable as an angiographic contrast agent, and a process for preparing the contrast agent.

### Technical Solution

To achieve the objects, the present invention provides a contrast agent including a compound of methoxy polyethylene glycol sulfhydryl (MeO-PEG-SH)-gold nanoparticle colloid, wherein the compound is prepared by reacting MeO-PEG-SH with a gold nanoparticle colloid, and the MeO-PEG-SH and the compound are respectively represented by Formulas 1 and 2 below.

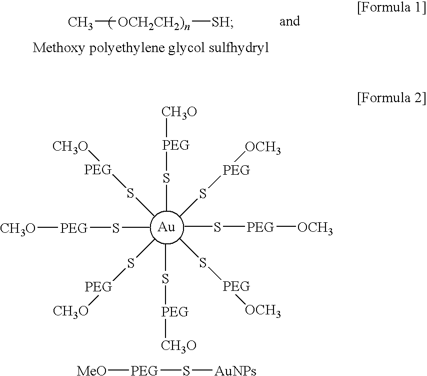

where MeO denotes methoxy, PEG denotes polyethylene glycol, S denotes sulfur, and AuNPs denotes gold nanoparticles.

Particles of the compound represented by Formula 2 may have a size in the range from 30 nm to 50 nm. For example, the particles of the compound represented by Formula 2 can have a size in the range from 35 nm to 40 nm. Therefore, the contrast agent can be suitably used as an angiographic contrast agent.

In another aspect of the present invention, there is provided a process for preparing a contrast agent, the method including: forming a gold nanoparticle colloid; and reacting the gold nanoparticle colloid with MeO-PEG-SH as shown by Formula 3 so as to obtain a compound of MeO-PEG-SH-gold nanoparticle colloid, wherein the MeO-PEG-SH and the compound are respectively represented by Formulas 1 and 2.

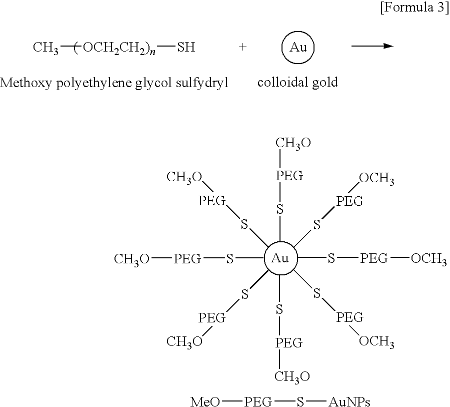

The gold nanoparticle colloid may be formed by changing Au³⁺ ions to Au⁰ particles. In this case, the Au³⁺ ions may be supplied by a HAuCl₄ compound, and sodium citrate may be used as a reducing agent for the Au³⁺ ions.

According to the present invention, the contrast agent formed of gold nanoparticle colloid coupled with PEG is nontoxic and capable of remaining in a blood vessel for a long time. Therefore, the contrast agent can be usefully used as a computed tomography (CT) contrast agent for angiography. Furthermore, the contrast agent is useful for inspecting blood vessel structures and new blood vessels in a tumor, and obtaining perfusion images. In addition, gold provides better visibility than iodine when used in an X-ray contrast agent. Therefore, better x-ray images can be obtained using the contrast agent of the present invention.

In the contrast agent of the present invention, colloid containing gold nanoparticles is coupled with PEG to make the colloid stable and increase the molecular weight of the gold nanoparticles. Thus, the contrast agent can remain in a blood vessel for a long time. Gold is a stable and nontoxic material sensitive to X-rays in CT. A PEG compound is easily dissolved in water because of its high oxygen concentration, so that the contrast agent containing the PEG compound can be hydrophilic and biocompatible. Since the contrast agent can be prepared in nano-sizes for imaging only blood vessels, the contrast agent can be usefully used for inspecting microstructures of blood vessels and new blood vessels in a tumor. Micro CT tests carried out using animals shows that the contrast agent of the present invention can be usefully used as a CT contrast agent. Since the contrast agent of the present invention is nontoxic to a human body, the contrast agent can be used for humans. That is, according to the present invention, an improved CT contrast agent can be provided.

According to the present invention, to prepare a contrast agent to be used for obtaining CT images of blood vessels of an organism and new blood vessels of a tumor, a gold nanoparticle colloid is coupled with MeO-PEG-SH in the way shown in Formula 3 as follows.

First, the gold nanoparticle colloid is prepared. In detail, the gold nanoparticle colloid has a red-wine color and is formed by reducing Au³⁺ ions to Au⁰ particles using a reducing agent such as sodium citrate.

Secondly, the gold nanoparticle colloid is coupled with MeO-PEG-SH. In detail, the gold nanoparticle colloid is reacted with PEG containing a -sulfhydryl (—SH) group to prepare a compound represented by Formula 2. Here, since sulfur atoms are easily coupled to gold (Au) atoms, the PEG containing the SH group is used as a capping agent.

The PEG is nontoxic and has a high oxygen concentration. Thus, the PEG is hydrophilic and is easily dissolved in water. Since the gold nanoparticle colloid is coupled with the PEG, the molecular weight of gold nanoparticles of the gold nanoparticle colloid increases. Thus, the gold particles can remain in a blood vessel for a longer time.

The gold nanoparticle colloid coupled with the MeO-PEG-SH has the color of a rich red wine. In ultraviolet-visible (UV-vis) spectrum, the gold nanoparticle colloid has a characteristic wavelength of about 510 nm, and the gold nanoparticle colloid coupled with the MeO-PEG-SH has a characteristic wavelength of about 520 nm. It can be understood that the absorbance peak of the gold nanoparticle colloid is shifted when gold nanoparticles of the gold nanoparticle colloid are capped with the PEG. In addition, the size and distribution of the gold nanoparticles of the gold nanoparticle colloid are measured using a dynamic light scattering (DLS) device and a transmission electron microscopy (TEM) device. The gold nanoparticle colloid coupled with the MeO-PEG-SH may have a pH in the range of 6.5 to 7.5. In this case, the gold nanoparticle colloid coupled with the MeO-PEG-SH can be biocompatible and biochemically stable.

### ADVANTAGEOUS EFFECTS

According to the present invention, a contrast agent is prepared by coupling a colloid containing gold nanoparticles with PEG to increase the biochemical stability and molecular weight of the gold nanoparticle colloid so that the contrast agent can remain in a blood vessel for a longer time. Gold is a stable and nontoxic material sensitive to X-rays in CT. A PEG compound is easily dissolved in water because of its high oxygen concentration, so that the contrast agent containing the PEG compound can be hydrophilic and biocompatible. Since the contrast agent can be prepared in nano-sizes for imaging only blood vessels, the contrast agent can be usefully used for inspecting microstructures of blood vessels and new blood vessels in a tumor. Micro CT tests carried out using animals shows that the contrast agent of the present invention can be usefully used as a CT contrast agent. Since the contrast agent of the present invention is nontoxic to a human body, the contrast agent can be used for humans. That is, according to the present invention, an improved CT contrast agent can be provided.

### DESCRIPTION OF DRAWINGS

FIG. 1 is a transmission electron microscopy (TEM) image of a gold nanoparticle colloid used as a precursor according to an embodiment of the present invention.

FIG. 2 is a TEM image of a compound expressed by Formula 2 according to an embodiment of the present invention.

FIG. 3 is a graph showing ultraviolet (UV) spectrums of the gold nanoparticle colloid and the formula-2 compound;

FIG. 4 is a dynamic light scattering (DLS) graph of the gold nanoparticle colloid.

FIG. 5 is a DSL graph of the formula-2 compound.

FIG. 6 illustrates micro computed tomography (CT) images of a mouse injected with the formula-2 compound.

### BEST MODE FOR CARRYING OUT THE INVENTION

The following will describe an embodiment of the present invention in more detail with reference to the accompanying drawings.

First, as a precursor for preparing a contrast agent of the present invention, a gold nanoparticle colloid was prepared as follows, according to an embodiment of the present invention.

\textcircled{1} 500 ml of 2.5×10⁻⁴ M HAuCl₄ aqueous solution was heated in a round-bottomed flask having a reflux condenser until the HAuCl₄ aqueous solution was refluxed.

\textcircled{2} When the HAuCl₄ aqueous solution was refluxed, 17.5 ml of 1% sodium citrate aqueous solution was added as a reducing agent to the HAuCl₄ aqueous solution to obtain a mixture having a molar ratio of 1:5. Here, the sodium citrate aqueous solution was added slowly but not too slowly.

\textcircled{3} Thereafter, the mixture was strongly agitated for 30 minutes for reaction.

\textcircled{4} After the reaction is completed, the mixture was cooled at room temperature.

\textcircled{5} The resulting product was inspected using a dynamic light scattering (DLS) device, a transmission electron microscopy (TEM) device, and an ultraviolet-visible (UV-vis) spectrophotometer.

Next, a contrast agent was prepared by coupling methoxy polyethylene glycol sulfhydryl (MeO-PEG-SH) and a gold nanoparticle colloid as follows, according to an embodiment of the present invention.

\textcircled{1} 50 ml of 1×10⁻³ M MeO-PEG-SH (MW 2,000) was added to 450 ml of gold nanoparticle colloid as a capping agent. Here, the MeO-PEG-SH was added slowly but not too slowly.

\textcircled{2} Thereafter, the mixture solution was strongly agitated for 12 hours for reaction.

\textcircled{3} After the reaction of the mixture solution was completed, the mixture solution was concentrated using a stirred cell to a volume 1/50 times the initial volume.

\textcircled{4} After that, the mixture solution was centrifuged at 10,000 rpm for 60 minutes using a high-speed centrifuge to a concentration of 1/1,000.

\textcircled{5} The resulting product was inspected using a DLS device, a TEM device, and a UV-vis spectrophotometer, and the concentration of gold in the resulting product was measured using an inductively coupled plasma mass spectrometry (ICP-MS) device.

In this way, a gold nanoparticle colloid coupled with MeO-PEG-SH was prepared.

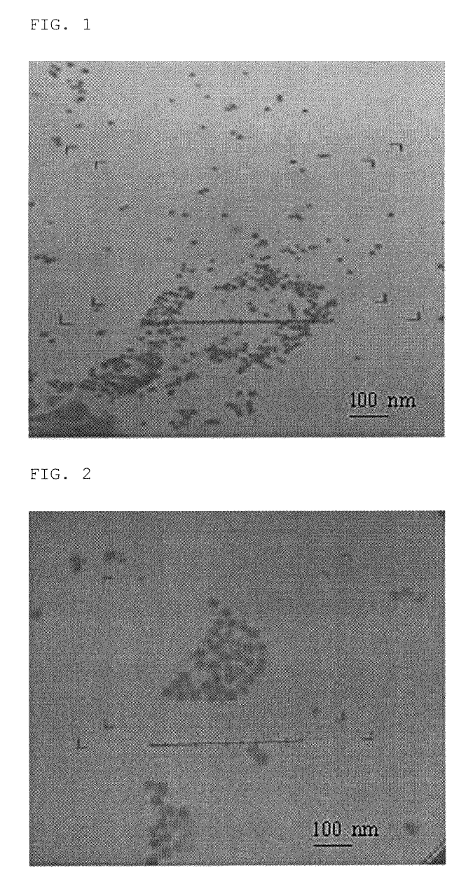

FIG. 1 is a TEM image of the gold nanoparticle colloid used as a precursor. The size of nanoparticles is approximately 10 nm, and the nanoparticles have a circular shape.

FIG. 2 is a TEM image of the gold nanoparticle colloid coupled with the MeO-PEG-SH. In this case, the size of the nanoparticles is approximately 35 nm to 40 nm, and the nanoparticles have a circular shape.

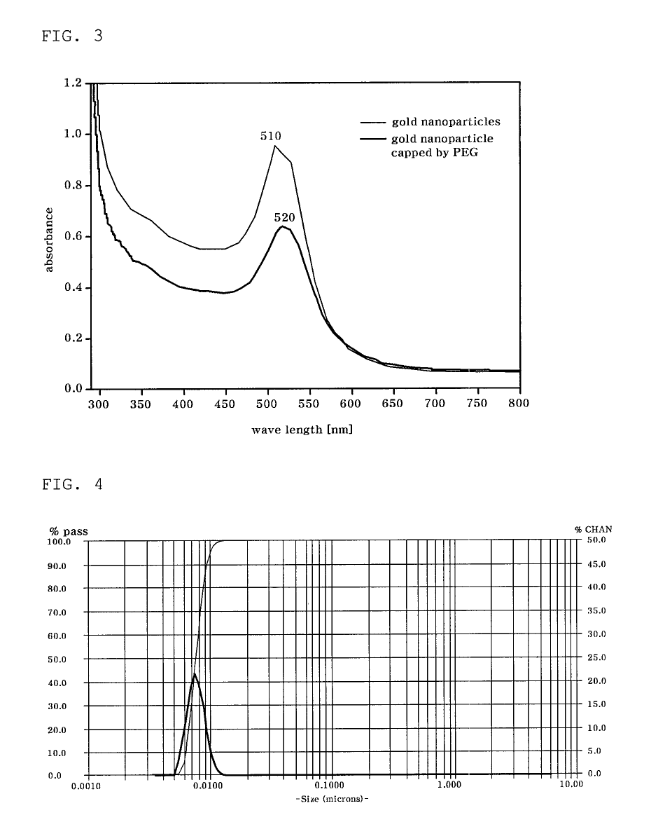

FIG. 3 is a graph showing UV-vis spectrums of the gold nanoparticle colloid before and after the gold nanoparticle colloid coupled with the MeO-PEG-SH. Before the gold nanoparticle colloid is coupled with the MeO-PEG-SH, the gold nanoparticle colloid has a characteristic wavelength of about 510 nm, and after the gold nanoparticle colloid is coupled with the MeO-PEG-SH, the gold nanoparticle colloid has a characteristic wavelength of about 520 nm. It can be understood that the absorbance peak of the gold nanoparticle colloid is shifted when the gold nanoparticles of the gold nanoparticle colloid are capped with the MeO-PEG-SH.

FIG. 4 illustrates a DLS graph of the gold nanoparticle colloid to show the distribution and size of the gold nanoparticles of the gold nanoparticle colloid. The size of the gold nanoparticles dispersed in the gold nanoparticle colloid is approximately 10 nm.

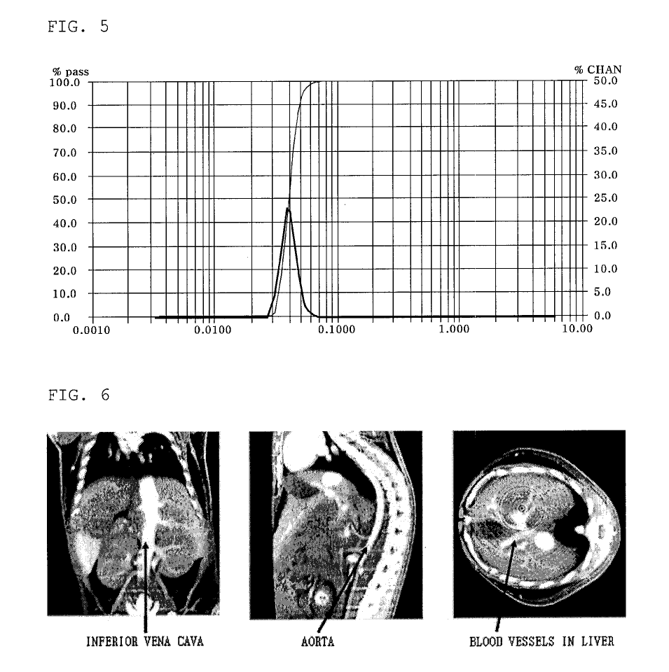

FIG. 5 illustrates a DLS graph of the gold nanoparticle coupled with the MeO-PEG-SH to show the distribution and size of the gold nanoparticles capped with the MeO-PEG-SH. The size of the gold nanoparticles, which are capped with the MeO-PEG-SH and dispersed in the gold nanoparticle colloid, is approximately 40 nm.

FIG. 6 illustrates micro computed tomography (CT) images of a mouse injected with the gold nanoparticle colloid coupled with the MeO-PEG-SH. Blood vessels of the mouse, such as the aorta, liver blood vessels, inferior vena cava, and heart blood vessels, are imaged as white as shown in FIG. 6.

1. A contrast agent comprising a compound of methoxy polyethylene glycol sulfhydryl (MeO-PEG-SH)-gold nanoparticle colloid, wherein the compound is prepared by reacting MeO-PEG-SH with a gold nanoparticle colloid, and the MeO-PEG-SH and the compound are respectively represented by Formulas 1 and 2 below: 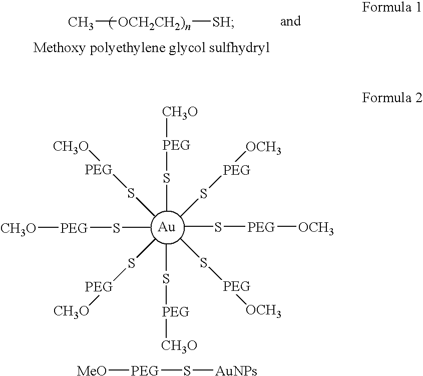
where MeO denotes methoxy, PEG denotes polyethylene glycol, S denotes sulfur, and AuNPs denotes gold nanoparticles.

2. The contrast agent of claim 1, wherein particles of the compound represented by Formula 2 have a size in a range from 30 nm to 50 nm.

3. The contrast agent of claim 1 or 2, the contrast agent is used for angiography.

4. A process for preparing a contrast agent, comprising:
forming a gold nanoparticle colloid; and
reacting the gold nanoparticle colloid with MeO-PEG-SH so as to obtain a compound of MeO-PEG-SH-gold nanoparticle colloid,
wherein the MeO-PEG-SH and the compound are respectively represented by Formulas 1 and 2 below: 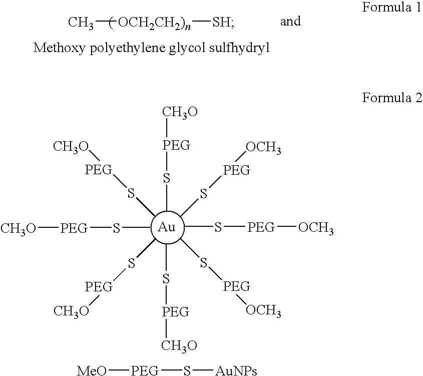
where MeO denotes methoxy, PEG denotes polyethylene glycol, S denotes sulfur, and AuNPs denotes gold nanoparticles.

5. The method of claim 4, wherein the gold nanoparticle colloid is formed by changing Au³⁺ ions to Au⁰ particles.

6. The method of claim 5, wherein the Au³⁺ ions are supplied by a HAuCl₄ compound, and sodium citrate is used as a reducing agent for the Au³⁺ ions.
